(19) United States
(12) Patent Application Publication
(10) Pub. No.: US 2025/0367153 A1
(43) Pub. Date: Dec. 4, 2025

(54) COMPOSITION AND APPLICATION THEREOF

(71) Applicant: SYNEURX INTERNATIONAL (TAIWAN) CORP., New Taipei City (TW)

(72) Inventors: Guo-Chuan TSAI, New Taipei City (TW); Wei-Hua CHANG, New Taipei City (TW); Lu-Ping LU, New Taipei City (TW); Chi-Sheng KUO, New Taipei City (TW); Xiao-Yi ZHUANG, New Taipei City (TW)

(73) Assignee: SYNEURX INTERNATIONAL (TAIWAN) CORP., New Taipei City (TW)

(21) Appl. No.: 19/220,905

(22) Filed: May 28, 2025

Related U.S. Application Data
(60) Provisional application No. 63/652,531, filed on May 28, 2024.

(51) Int. Cl. A61K 31/198; A61K 31/047; A61K 31/185; A61K 31/19; A61K 31/192; A61K 31/197; A61K 31/202; A61K 31/405; A61K 31/42; A61K 31/455; A61K 31/685; A61K 31/7034; A61K 35/00; A61K 35/745; A61K 35/747; A61K 36/53; A61K 36/54; A61K 36/9066; A61P 25/20; A61P 25/24; A61P 25/28

(52) U.S. Cl. CPC A61K 31/198; A61K 31/047; A61K 31/185; A61K 31/19; A61K 31/192; A61K 31/197; A61K 31/202; A61K 31/405; A61K 31/42; A61K 31/455; A61K 31/685; A61K 31/7034; A61K 35/745; A61K 35/747; A61K 36/53; A61K 36/54; A61K 36/9066; A61P 25/20; A61P 25/24; A61P 25/28; A61K 2035/115

(57) ABSTRACT

A composition, comprising a D-amino acid oxidase (DAAO) inhibitor and a N-methyl-D-aspartate (NMDA) modulator. Also provided herein are the method of the uses of by administering to a subject in need thereof an effective amount of the composition.

### CROSS-REFERENCE TO RELATED APPLICATIONS

This non-provisional application claims priority under 35 U.S.C. § 119 (a) on Patent Application No. 63/652,531 filed in U.S.A. on May 28, 2024, the entire contents of which are hereby incorporated by reference.

### BACKGROUND

### 1. Technical Field

This disclosure relates to a composition for treating, preventing, or delaying the onset of psychiatric disorders or central nervous system disorders. Furthermore, this disclosure relates to a composition for improving sense, mood, sleeping behavior, anxiety, depression, concentration, attention, cognition, longevity, thought, or memory.

### 2. Related Art

The frontal lobe is located at the front of each cerebral hemisphere and play a major role in cognitive functions such as attention, working memory, creative and critical thinking, planning, decision making, inhibitory control, and emotional regulation. N-methyl-D-aspartate (NMDA) receptor is a subtype glutamatergic receptor that plays a critical role in cognition, memory, and neurotoxicity. Regulation of NMDA receptor is suggested to be beneficial for treating diseases involved in frontal lobe dysfunction, such as cognitive deficits and diseases of the central nervous system. D-amino acid oxidase (DAAO) is a peroxisomal enzyme that oxidizes D-amino acids to the corresponding imino acids. It has been reported that DAAO is involved in the metabolism of brain D-amino acids and the regulation of the glutamatergic neurotransmission which the central nervous system disorders are associated with. As such, NMDA or DAAO can be targets for treating cognitive deficits or diseases of the central nervous system.

### SUMMARY

The present disclosure is based, at least in part, on the unexpected discovery that a composition comprising a DAAO inhibitor and a NMDA modulator exerts potentiated therapeutic effects on the psychiatric disorder or the central nervous system (CNS) disorder.

According to one or more embodiment, the present disclosure provides a composition, comprising: a D-amino acid oxidase (DAAO) inhibitor; and a N-methyl-D-aspartate (NMDA) modulator.

According to one or more embodiment, the present disclosure provides a composition suitable for improving sense, mood, sleeping behavior, anxiety, depression, concentration, attention, cognition, longevity, thought, or memory, or for treating, preventing, or delaying the onset of dementia, mild cognitive impairments, or memory deficit, wherein the composition comprises an effective amount of a D-amino acid oxidase (DAAO) inhibitor and an effective amount of a N-methyl-D-aspartate (NMDA) modulator.

According to one or more embodiment, the present disclosure provides a composition suitable for treating, preventing, or delaying the onset of serotonin-or melatonin-mediated disorders, wherein the composition comprises an effective amount of a D-amino acid oxidase (DAAO) inhibitor and an effective amount of a N-methyl-D-aspartate (NMDA) modulator.

According to one or more embodiment, the present disclosure provides a combination use for treating, preventing, or delaying the onset of dementia or mild cognitive impairments, comprising administering an effective amount of a D-amino acid oxidase (DAAO) inhibitor and an effective amount of a N-methyl-D-aspartate (NMDA) modulator to a subject in need thereof.

According to one or more embodiment, the present disclosure provides a method for treating, preventing, or delaying the onset of psychiatric disorder, comprising administering an effective amount of a D-amino acid oxidase (DAAO) inhibitor and an effective amount of a N-methyl-D-aspartate (NMDA) modulator to a subject in need thereof.

According to one or more embodiment, the present disclosure provides a method for treating, preventing, or delaying the onset of central nervous system (CNS) disorder by administering an effective amount of a D-amino acid oxidase (DAAO) inhibitor and an effective amount of a N-methyl-D-aspartate (NMDA) modulator to a subject in need thereof.

### BRIEF DESCRIPTION OF THE DRAWINGS

The present disclosure will become better understood from the detailed description given hereinbelow and the accompanying drawings which are given by way of illustration only and thus are not intended to limit the present disclosure and wherein:

FIG. 1 shows HPLC chromatograms of cinnamon bark extracts purchased from (A) Supplier A (Cinnamomum cassia from China), (B) Supplier B (Cinnamomum cassia from China), (C) Supplier C (Cinnamomum cassia from India), and (D) Supplier D (Cinnamomum Burmannii from China), and cinnamon leaf powder purchased from (E) Supplier E (Cinnamomum osmophloeum from Taiwan), and HPLC chromatogram of (F) mixed standard solution of CM, CA and TCAL (15.63 μg/mL). Peaks: (CM) coumarin, (CA) cinnamic acid, and (TCAL) trans-cinnamaldehyde;

FIG. 2 shows the discrimination index of novel object recognition test in MK801-treated mice received acute administration (50 or 100 mg/kg) of NMDA modulator alone, DAAO inhibitor alone, or NMDA modulator combined with DAAO inhibitor. Data were presented as mean±SEM and analyzed with Student's T-test. * P<0.05, ***P<0.001, compared with MK-801 alone group;

FIG. 3 shows the discrimination index of novel object recognition test in MK801-treated mice received acute administration of NMDA modulator, cinnamon extract (CE) or NMDA modulator combined with cinnamon extract. Data were presented as mean±SEM and analyzed with Student's T-test. *P<0.05, compared with MK-801 alone group;

FIG. 4 shows the immobility of forced-swimming test in control and test-groups, including NMDA modulator, DAAO inhibitor, and NMDA modulator+DAAO inhibitor combination-treated mice. Combination treatment significantly reduces the immobility time in FST, an indicator of depressed moods, compared with the vehicle group (*P<0.05) and NMDA modulator-treated (#P<0.05) group in this experiment;

FIG. 5 shows the effects of NMDA modulator combined with DAAO inhibitor and NMDA modulator combined with cinnamon extract on prepulse inhibition test in MK-801 (0.3 mg/kg; intraperitoneal injection)-treated mice. Data were presented as mean±SEM and analyzed with Student's T-test. ** P<0.001, compared with MK-801 alone group;

FIG. 6 shows the sedative effects of NMDAM and DAAOI in mice. Locomotor activity following acute administration of NMDA modulator (NMDAM) combined with D-amino acid oxidase inhibitors (DAAOI). Data were presented as mean±SEM and analyzed with Student's T-test. Statistical significance was marked as follows: * P<0.05, compared with vehicle group;

FIG. 7 shows the discrimination index in the novel object recognition test in MK-801-insulted mice following acute administration of combined NMDA modulator (NMDAM) and D-amino acid inhibitor (DAAOI) treatment, or NMDAM and DAAOI alone; and

FIG. 8 shows therapeutic effects of NMDAM and DAAOI in sleep disturbance. Four weeks of combined NMDAM and DAAOI treatment significantly reduced PSQI scores in patients with sleep disturbance. Data were presented as mean±SEM and analyzed with Student's T-test. Statistical significance was marked as follows: * P<0.05, compared with the pre-treatment score.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

As used herein, the term “composition” and “formulation” are used interchangeably.

As used herein, the term “treating” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

As used herein, “onset” of a target disease or disorder includes initial onset and/or recurrence.

As used herein, the term “administer”, “administering”, or “administration” refers to implanting, absorbing, ingesting, injecting, inhaling, or otherwise introducing a composition described herein, in or on a subject.

As used herein, the terms “disease” and “disorder” are used interchangeably.

The terms “subject” and “individual” are used interchangeably herein and refer to a mammal being assessed for ability and behavior improvement. The term “patient” refers to a mammal being assessed for treatment and/or being treated. Subjects may be human, but also include other mammals, particularly those mammals useful as laboratory models for human disease, e.g., mouse, rat, rabbit, dog, etc. When the ability degradation or behavior deterioration reaches the level which would become a symptom of a disease or disorder, the subject/individual with the ability or behavior may be a patient suffering from the disease or disorder.

As used herein, the term “an effective amount” refers to the amount of an active agent required to confer ability/behavior improvement on the subject/individual or therapeutic effect on the patient.

As used herein, the term “pharmaceutically acceptable” refers to which are, within the scope of sound medical judgment, suitable for use in humans and mammals without undue toxicity, irritation, allergic response, and the like, and are commensurate with a reasonable benefit/risk ratio.

Unless otherwise specified, amino acids described herein can include those with various isomeric forms, e.g., D and L enantiomers and racemic mixtures. For example, serine described herein includes L-serine, D-serine, and DL-serine. Unless otherwise specified, DL-amino acid described herein refers a racemic mixture, a mixture of equal quantities of two enantiomers, or substances that have dissymmetric molecular structures that are mirror images of one another. Taking serine as an example, DL-serine described herein includes equal quantities of D-serine and L-serine.

As used herein, the term “combination use” is used interchangeably with the term “combination therapy” herein and embraces administration of two or more agents in a sequential manner or a simultaneous manner. The agents may be administered by the same route or by different routes.

As used herein, the term “parenteral” includes subcutaneous, intracutaneous, intravenous, intramuscular, intraarticular, intraarterial, intrasynovial, intrasternal, intrathecal, intralesional, and intracranial injection or infusion techniques.

The present disclosure provides a composition comprising a D-amino acid oxidase (DAAO) inhibitor and a N-methyl-D-aspartate (NMDA) modulator. The DAAO inhibitor can inhibit DAAO, thereby improving basic functioning, body weight, hyperactivity, anxiety, depression, suicidal ideation and/or behavior, sensorimotor gating, pain threshold, memory and cognitive behaviors. The NMDA receptor is a critical component of the glutamatergic system in the brain, playing a key role in synaptic plasticity, memory formation, and neural communication. Modulating the NMDA receptor can influence neuronal excitability and has therapeutic potential for a variety of neurological and psychiatric disorders, such as Alzheimer's disease, major depressive disorder, and schizophrenia. NMDA modulators can either enhance or inhibit receptor function, providing a targeted approach to restore balance in pathological conditions where NMDA receptor activity is disrupted.

The DAAO inhibitor may be selected from a group consisting of cinnamon extract, sodium benzoate, lithium benzoate, benzoic acid, sorbic acid, cinnamic acid, and tannic acid.

The cinnamon extract may be extracted from bark or leaf of Cinnamomum cassia, Cinnamomum austrosinense, Cinnamomum brevipedunculatum, Cinnamomum burmannii, Cinnamomum insularimontanum, Cinnamomum kotoense, Cinnamomum macrostemon, Cinnamomum osmophloeum, Cinnamomum reticulatum, Cinnamomum sericeum, Cinnamomum subavenium, Cinnamomum temuuifolium nervosum, Cinnamomum verum, or Cinnamomum loureiroi.

The cinnamon extract may comprise cinnamic acid, cinnamaldehyde, cinnamyl acetate, or cinnamyl alcohol.

The NMDA modulator may be selected from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, and DL-cycloserine, a probiotic composition, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and tumeric extract.

The probiotic composition may comprise a plurality of probiotic strains selected from the group consisting of Lactobacillus acidophilus, Lactobacillus casei, Lactobacillus rhamnosus, Lactobacillus plantarum, Lactobacillus reuteri, Lactobacillus fermentum, Lactobacillus salivarius, Lactobacillus bulgaricus, Lactobacillus brevis, Lactobacillus paracasei, Lactobacillus gasseri, Lactobacillus johnsonii, Bacillus coagulans, Bifidobacterium adolescentis, Bifidobacterium animalis subsp. lactis, Bifidobacterium bifidum, Bifidobacterium breve, Bifidobacterium longum, Bifidobacterium infantis, Bifidobacterium lactis, Bifidobacterium pseudocatenulatum, Bifidobacterium thermophilum, Bifidobacterium pseudolongum or Clostridium buyricum.

In some embodiments, the probiotic count may be 1×10⁶ to 1×10¹² CFU, but the present disclosure is not limited thereto. In other embodiments, the probiotic count may be 1×10⁹ to 1×10¹¹ CFU, but the present disclosure is not limited thereto.

In some embodiments, the weight ratio of the DAAO inhibitor to the NMDA modulator in the composition may be 1:100 to 100:1, but the present disclosure is not limited thereto. In other embodiments, the weight ratio of the DAAO inhibitor to the NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of the DAAO inhibitor to the NMDA modulator in the composition may be 5:1, 4:1, 3:1, 2:1, 1:1, 1:2, 1:3, 1:4, or 1:5.

In some embodiments, the DAAO inhibitor may be cinnamon extract, sodium benzoate, lithium benzoate, benzoic acid, sorbic acid, cinnamic acid, or tannic acid, and the NMDA modulator may be glycine, sarcosine, dimethylglycine (DMG), DL-serine, DL-alanine, DL-cycloserine, a probiotic composition, taurine, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, or tumeric extract, wherein the weight ratio of DAAO inhibitor to NMDA modulator in the composition may be 1:100 to 100:1, but the present disclosure is not limited thereto. In other embodiments, the weight ratio of DAAO inhibitor to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator in the composition may be 5:1, 4:1, 3:1, 2:1, 1:1, 1:2, 1:3, 1:4, or 1:5.

In some embodiments, the DAAO inhibitor may be cinnamon extract, sodium benzoate, cinnamic acid, or tannic acid, and the NMDA modulator may be glycine, sarcosine, dimethylglycine (DMG), DL-serine, DL-alanine, or DL-cycloserine, wherein the weight ratio of DAAO inhibitor to NMDA modulator in the composition may be 1:100 to 100:1, but the present disclosure is not limited thereto. In other embodiments, the weight ratio of DAAO inhibitor to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator in the composition may be 5:1, 4:1, 3:1, 2:1, 1:1, 1:2, 1:3, 1:4, or 1:5.

In some embodiments, the DAAO inhibitor may be cinnamon extract, sodium benzoate, cinnamic acid, or tannic acid, and the NMDA modulator may be glycine, sarcosine, dimethylglycine (DMG), D-serine, D-alanine, or D-cycloserine, wherein the weight ratio of DAAO inhibitor to NMDA modulator in the composition may be 1:100 to 100:1, but the present disclosure is not limited thereto. In other embodiments, the weight ratio of DAAO inhibitor to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator in the composition may be 5:1, 4:1, 3:1, 2:1, 1:1, 1:2, 1:3, 1:4, or 1:5.

In some embodiments, the DAAO inhibitor may be cinnamon extract, and the NMDA modulator may be glycine, sarcosine, dimethylglycine (DMG), DL-serine, DL-alanine, or DL-cycloserine, wherein the weight ratio of cinnamon extract to NMDA modulator in the composition may be 1:100 to 100:1, but the present disclosure is not limited thereto. In other embodiments, the weight ratio of cinnamon extract to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of cinnamon extract to NMDA modulator in the composition may be 5:1, 4:1, 3:1, 2:1, 1:1, 1:2, 1:3, 1:4, or 1:5.

In some embodiments, the DAAO inhibitor may be cinnamic acid, and the NMDA modulator may be glycine, sarcosine, dimethylglycine (DMG), D-serine, D-alanine, or D-cycloserine, wherein the weight ratio of cinnamic acid to NMDA modulator in the composition may be 1:100 to 100:1, but the present disclosure is not limited thereto. In other embodiments, the weight ratio of cinnamic acid to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of cinnamic acid to NMDA modulator in the composition may be 5:1, 4:1, 3:1, 2:1, 1:1, 1:2, 1:3, 1:4, or 1:5.

In some embodiments, the DAAO inhibitor may be sodium benzoate, and the NMDA modulator may be glycine, sarcosine, dimethylglycine (DMG), D-serine, D-alanine, or D-cycloserine, wherein the weight ratio of sodium benzoate to NMDA modulator in the composition may be 1:100 to 100:1, but the present disclosure is not limited thereto. In other embodiments, the weight ratio of sodium benzoate to NMDA modulator in the composition may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of sodium benzoate to NMDA modulator in the composition may be 5:1, 4:1, 3:1, 2:1, 1:1, 1:2, 1:3, 1:4, or 1:5.

In some embodiments, the composition may comprise at least 50 mg of the DAAO inhibitor and at least 50 mg of NMDA modulator, but the present disclosure is not limited thereto.

In some embodiments, the composition may further comprise a pharmaceutically acceptable excipient, carrier, diluent, binder, additive, filler, lubricant, nutrient, or a mixture thereof. Some of the above additions may provide convenience for the subsequent processing of the composition without deteriorating the improvement effect or therapeutic effect of the composition. Some of the above additions may also enhance one or more properties of the composition, e.g., bioactivity, stability, bioavailability, and other pharmacokinetics and/or bioactivities.

In some embodiments, the composition may be a pharmaceutical composition, a nutraceutical composition, a medical food, a health food, a health supplement, or a food additive. The form of the final product does not limit the composition of the present disclosure. The composition according to the present disclosure may have different names for different applications/usages.

According to the present invention, the composition can be in the form of a food additives (an exemplary example of a food composition), which can be added to an edible material to prepare a food product for human or animal consumption. According to the present invention, examples of the food product may include, but are not limited to: fluid milk products, such as milk and concentrated milk; fermented milk, such as yogurt (yogurt), sour milk and frozen yogurt; milk powder; ice cream; cream cheeses; dry cheeses; soybean milk; fermented soybean milk; vegetable-fruit juices; fruit juices; sports drinks; energy drinks, confectionery; jelly; candies; jelly candies; health foods; animal feeds; and dietary supplements.

In some embodiments, the composition may be formulated in a form of tablet, pill, capsule, powder, granule, solution, suspension, soft chew, or gel. The formulation does not limit the composition of the present disclosure and may be determined depending on the final product, using occasion, or timing of use.

The present disclosure also provides a method for treating, preventing, or delaying the onset of psychiatric disorder or central nervous system (CNS) disorder, the method comprising administering to a subject in need thereof an effective amount of any of the compositions described herein. In some embodiments, the effective amount of the composition comprises an effective amount of a DAAO inhibitor and an effective amount of a NMDA modulator. In other embodiments, any of the compositions described herein can be a pharmaceutical composition, which further comprises a pharmaceutically acceptable carrier. In other embodiments, the composition can be a healthy food product (e.g., a nutraccutical composition, a medical food, or a health food), which may comprise an edible carrier.

In some embodiments, the DAAO inhibitor may be selected from a group consisting of cinnamon extract, sodium benzoate, lithium benzoate, benzoic acid, sorbic acid, cinnamic acid, and tannic acid. The cinnamon extract may be extracted from bark or leaf of Cinnamomum cassia, Cinnamomum austrosinense, Cinnamomum brevipedunculatum, Cinnamomum burmannii, Cinnamomum insularimontanum, Cinnamomum kotoense, Cinnamomum macrostemon, Cinnamomum osmophloeum, Cinnamomum reticulatum, Cinnamomum sericeum, Cinnamomum subavenium, Cinnamomum temutifolium nervosum, Cinnamomum verum, or Cinnamomum loureiroi. The NMDA modulator may be selected from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, and DL-cycloserine, a probiotic composition, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and turmeric extract. The probiotic composition may comprise a plurality of probiotic strains selected from the group consisting of Lactobacillus acidophilus, Lactobacillus casei, Lactobacillus rhamnosus, Lactobacillus plantarum, Lactobacillus reuteri, Lactobacillus fermentum, Lactobacillus salivarius, Lactobacillus bulgaricus, Lactobacillus brevis, Lactobacillus paracasei, Lactobacillus gasseri, Lactobacillus johnsonii, Bacillus coagulans, Bifidobacterium adolescentis, Bifidobacterium animalis subsp. Lactis, Bifidobacterium bifidum, Bifidobacterium breve, Bifidobacterium longum, Bifidobacterium infantis, Bifidobacterium lactis, Bifidobacterium pseudocatenulatum, Bifidobacterium thermophilum, Bifidobacterium pseudolongum or Clostridium buyricum.

Psychiatric disorder is a behavioral or mental pattern that causes significant distress or impairment of personal functioning. The method according to an embodiment of the present disclosure can treat, prevent, or delay the onset of the psychiatric disorder comprising but not limited to depressive disorder, major depression, anhedonia, eating disorder, elimination disorder, feeding disorder, depression, dysthymia, mood disorder, anxiety disorder, personality disorder, addictive behavior, obsessive-compulsive and related disorder, trauma- and stressor-related disorders, dissociative disorder, sleep-wake disorder, sleep disorder, insomnia, somatic symptom and related disorder, social anxiety disorder, depression-related syndrome, symptom of depression due to a physical disorder, and drug-induced symptom of depression.

The method according to an embodiment of the present disclosure can treat, prevent, or delay the onset of the psychiatric disorder selected from a group consisting of depressive disorder, major depression, anhedonia, eating disorder, elimination disorder, feeding disorder, depression, dysthymia, mood disorder, anxiety disorder, personality disorder, addictive behavior, obsessive-compulsive and related disorder, trauma-and stressor-related disorders, dissociative disorder, sleep-wake disorder, sleep disorder, insomnia, somatic symptom and related disorder, social anxiety disorder, depression-related syndrome, symptom of depression due to a physical disorder, and drug-induced symptom of depression.

CNS disorder is a group of neurological disorders that affect the structure or function of the brain or spinal cord, which collectively form the central nervous system. The method according to an embodiment of the present disclosure can treat, prevent, or delay the onset of the CNS disorder comprising but not limited to schizophrenia, bipolar disorder (BD), attention-deficit disorder, attention-deficit hyperactivity disorder (ADHD), obsessive compulsive disorder (OCD), Tourette's syndrome, blepharospasm, post-traumatic stress disorder (PTSD), addiction disorder, psychotic disorder, panic disorder, autism spectrum disorder (ASD), Asperger's disorder, Alzheimer's disease, mild cognitive disorder (MCI), benign forgetfulness, vascular dementia, dementia with Lewy bodies (DLB), Huntington's disease (HD), premenstrual syndrome, nocturnal enuresis, non-epileptic seizures, mild cognitive impairment, mania, learning disorder, pain, Parkinson's disease, Duchenne muscular dystrophy, stoke, closed head injury, memory deficits, frontotemporal dementia (FTD), multiple sclerosis, amyotrophic lateral sclerosis (ALS), Lyme borreliosis, tick-borne diseases (TBD), fragile X syndrome (FXS), and traumatic brain injury.

The method according to an embodiment of the present disclosure can treat, prevent, or delay the onset of the CNS disorder selected from a group consisting of schizophrenia, bipolar disorder (BD), attention-deficit disorder, attention-deficit hyperactivity disorder (ADHD), obsessive compulsive disorder (OCD), Tourette's syndrome, blepharospasm, post-traumatic stress disorder (PTSD), addiction disorder, psychotic disorder, panic disorder, autism spectrum disorder (ASD), Asperger's disorder, Alzheimer's disease, mild cognitive disorder (MCI), benign forgetfulness, vascular dementia, dementia with Lewy bodies (DLB), Huntington's disease (HD), premenstrual syndrome, nocturnal enuresis, non-epileptic seizures, mild cognitive impairment, mania, learning disorder, pain, Parkinson's disease, Duchenne muscular dystrophy, stoke, closed head injury, memory deficits, frontotemporal dementia (FTD), multiple sclerosis, amyotrophic lateral sclerosis (ALS), Lyme borreliosis, tick-borne diseases (TBD), fragile X syndrome (FXS), and traumatic brain injury.

The method according to an embodiment of the present disclosure can treat, prevent, or delay the onset of the CNS disorder selected from a group consisting of attention-deficit disorder, attention-deficit hyperactivity disorder (ADHD), dementia, mild cognitive disorder (MCI), and learning disorder.

The present disclosure also provides a method or a combination use for treating, preventing, or delaying the onset of dementia, mild cognitive impairments, memory deficit, for improve sense, mood, sleeping behavior, anxiety, depression, concentration, attention, cognition, longevity, thought or memory, and can also treat, prevent, or delay the onset of serotonin- or melatonin-mediated disorders, comprising administering an effective amount of a DAAO inhibitor and an effective amount of a NMDA modulator to a subject in need thereof.

The term “combination use” embraces administration of two or more agents in a sequential manner or a simultaneous manner. The agents may be administered by the same route or by different routes. For example, the DAAO inhibitor and the NMDA modulator may be administrated sequentially or simultaneously and may be administrated by oral or parenteral. The DAAO inhibitor may be selected from a group consisting of cinnamon extract, sodium benzoate, lithium benzoate, benzoic acid, sorbic acid, cinnamic acid, and tannic acid. The cinnamon extract may be extracted from bark or leaf of Cinnamomum cassia, Cinnamomum austrosinense, Cinnamomum brevipedunculatum, Cinnamomum burmannii, Cinnamomum insularimontamum, Cinnamomum kotoense, Cinnamomum macrostemon, Cinnamomum osmophloeum, Cinnamomum reticulatum, Cinnamomum sericeum, Cinnamomum subavenium, Cinnamomum temufolium nervosum, Cinnamomum verum, or Cinnamomum loureiroi. The NMDA modulator may be selected from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, and DL-cycloserine, a probiotic composition, taurine, nicotinamide, tryptophan, threonine, GABA, tyrosine, 5HTP, DHA, EPA, lutein, lemon balm extract, and turmeric extract. The probiotic composition may comprise a plurality of probiotic strains selected from the group consisting of Lactobacillus acidophilus, Lactobacillus casei, Lactobacillus rhamnosus, Lactobacillus plantarum, Lactobacillus reuteri, Lactobacillus fermentum, Lactobacillus salivarius, Lactobacillus bulgaricus, Lactobacillus brevis, Lactobacillus paracasei, Lactobacillus gasseri, Lactobacillus johnsonii, Bacillus coagulans, Bifidobacterium adolescentis, Bifidobacterium animalis subsp. Lactis, Bifidobacterium bifidum, Bifidobacterium breve, Bifidobacterium longum, Bifidobacterium infantis, Bifidobacterium lactis, Bifidobacterium pseudocatenulatum, Bifidobacterium thermophilum, Bifidobacterium pseudolongum or Clostridium buyricum.

In some embodiments, the weight ratio of the DAAO inhibitor to the NMDA modulator may be 1:100 to 100:1, but the present disclosure is not limited thereto. In other embodiments, the weight ratio of the DAAO inhibitor to the NMDA modulator may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of the DAAO inhibitor to the NMDA modulator may be 5:1, 4:1, 3:1, 2:1, 1:1, 1:2, 1:3, 1:4, or 1:5.

In some embodiments, the DAAO inhibitor may be cinnamon extract, sodium benzoate, lithium benzoate, benzoic acid, sorbic acid, cinnamic acid, and tannic acid, and the NMDA modulator may be glycine, sarcosine, dimethylglycine (DMG), DL-serine, DL-alanine, DL-cycloserine, a probiotic composition, taurine, tryptophan, threonine, GABA, tyrosine, 5HTP, DHA, EPA, lutein, lemon balm extract, or turmeric extract, wherein the weight ratio of DAAO inhibitor to NMDA modulator may be 1:100 to 100:1, but the present disclosure is not limited thereto. In other embodiments, the weight ratio of DAAO inhibitor to NMDA modulator may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator may be 5:1, 4:1, 3:1, 2:1, 1:1, 1:2, 1:3, 1:4, or 1:5.

In some embodiments, the DAAO inhibitor may be cinnamon extract, sodium benzoate, cinnamic acid, and tannic acid, and the NMDA modulator may be glycine, sarcosine, dimethylglycine (DMG), DL-serine, DL-alanine, or DL-cycloserine, wherein the weight ratio of DAAO inhibitor to NMDA modulator may be 1:100 to 100:1, but the present disclosure is not limited thereto. In other embodiments, the weight ratio of DAAO inhibitor to NMDA modulator may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator may be 5:1, 4:1, 3:1, 2:1, 1:1, 1:2, 1:3, 1:4, or 1:5.

In some embodiments, the DAAO inhibitor may be cinnamon extract, sodium benzoate, cinnamic acid, and tannic acid, and the NMDA modulator may be glycine, sarcosine, dimethylglycine (DMG), D-serine, D-alanine, or D-cycloserine, wherein the weight ratio of DAAO inhibitor to NMDA modulator may be 1:100 to 100:1, but the present disclosure is not limited thereto. In other embodiments, the weight ratio of DAAO inhibitor to NMDA modulator may be 1:90 to 90:1, 1:80 to 80:1, 1:70 to 70:1, 1:60 to 60:1, 1:50 to 50:1, 1:40 to 40:1, 1:30 to 30:1, 1:20 to 20:1, 1:10 to 10:1, 1:6 to 6:1, 1:5 to 5:1, 1:3 to 3:1, or 1:2 to 2:1. In other embodiments, the weight ratio of DAAO inhibitor to NMDA modulator may be 5:1, 4:1, 3:1, 2:1, 1:1, 1:2, 1:3, 1:4, or 1:5

In some embodiments, the effective amount of DAAO inhibitor may be at least 10 mg, and the effective amount of NMDA modulator may be at least 10 mg. That is, the administration to a subject in need thereof may comprise at least 10 mg of DAAO inhibitor and at least 10 mg of NMDA modulator.

In some embodiments, the DAAO inhibitor may be cinnamon extract, sodium benzoate, cinnamic acid, or tannic acid, and the NMDA modulator may be glycine, sarcosine, dimethylglycine (DMG), D-serine, D-alanine, or D-cycloserine, wherein the effective amount of the DAAO inhibitor may be 10 mg to 400000 mg, and the effective amount of the NMDA modulator may be 10 mg to 200000 mg. That is, the administration to a subject in need thereof may comprise 10 mg to 400000 mg of DAAO inhibitor and 10 mg to 200000 mg of NMDA modulator. In another embodiment, the effective amount may be 100 mg to 100000 mg of DAAO inhibitor and 100 mg to 100000 mg of NMDA modulator. In yet another embodiment, the effective amount may be 200 mg to 50000 mg of DAAO inhibitor and 200 mg to 50000 mg of NMDA modulator.

In some embodiments, the DAAO inhibitor may be cinnamon extract, sodium benzoate, cinnamic acid, or tannic acid, and the NMDA modulator may be glycine, sarcosine, dimethylglycine (DMG), DL-serine, DL-alanine, or DL-cycloserine, wherein the effective amount of the DAAO inhibitor is 10 mg to 400000 mg, and the effective amount of the NMDA modulator is 10 mg to 400000 mg. That is, the administration to a subject in need thereof may comprise 10 mg to 400000 mg of DAAO inhibitor and 10 mg to 400000 mg of NMDA modulator. In another embodiment, the effective amount may be 100 mg to 100000 mg of DAAO inhibitor and 100 mg to 100000 mg of NMDA modulator. In yet another embodiment, the effective amount may be 200 mg to 50000 mg of DAAO inhibitor and 200 mg to 50000 mg of NMDA modulator.

In some embodiments, for treating, preventing, or delaying the onset of psychiatric disorder, the DAAO inhibitor may be cinnamon extract, sodium benzoate, cinnamic acid, or tannic acid, and the NMDA modulator may be glycine, sarcosine, dimethylglycine (DMG), D-serine, D-alanine, or D-cycloserine, wherein the effective amount of the DAAO inhibitor may be 200 mg/day to 10 g/day, and the effective amount of the NMDA modulator is 200 mg/day to 10 g/day.

In some embodiments, for treating, preventing, or delaying the onset of psychiatric disorder, the DAAO inhibitor may be cinnamon extract, sodium benzoate, cinnamic acid, or tannic acid, and the NMDA modulator may be glycine, sarcosine, dimethylglycine (DMG), DL-serine, DL-alanine, or DL-cycloserine, wherein the effective amount of the DAAO inhibitor may be 200 mg/day to 10 g/day, and the effective amount of the NMDA modulator may be 400 mg/day to 20 g/day.

In some embodiments, for treating, preventing, or delaying the onset of CNS disorder, the DAAO inhibitor may be cinnamon extract, sodium benzoate, cinnamic acid, or tannic acid, and the NMDA modulator may be glycine, sarcosine, dimethylglycine (DMG), D-serine, D-alanine, or D-cycloserine, wherein the effective amount of the DAAO inhibitor may be 200 mg/day to 10 g/day, and the effective amount of the NMDA modulator may be 200 mg/day to 10 g/day.

In some embodiments, for treating, preventing, or delaying the onset of CNS disorder, the DAAO inhibitor may be cinnamon extract, sodium benzoate, cinnamic acid, or tannic acid, and the NMDA modulator may be glycine, sarcosine, dimethylglycine (DMG), DL-serine, DL-alanine, or DL-cycloserine, wherein the effective amount of the DAAO inhibitor may be 200 mg/day to 10 g/day, and the effective amount of the NMDA modulator may be 400 mg/day to 10 g/day.

In some embodiments, the administration may be by oral or parenteral. In some embodiments, the administration may be one time per day to one time per month.

In some embodiments, the subject is on another CNS disorder treatment. The therapeutic agent used in another CNS disorder treatment is selected from the additional therapeutic agent described herein.

In some examples, the additional therapeutic agent is an agent for treating a CNS disease/disorder. Such a therapeutic agent may be an antipsychotic drug. Exemplary antipsychotic drugs include, but are not limited to, butyrophenone (e.g., Haloperidol (HALDOL™), phenothiazine (e.g., chlorpromazine (THORAZINE™), fluphenazine (PROLIXIN™), perphenazine (TRILAFON™), prochlorperazine (COMPAZINE™), thioridazine (MELLARIL™), trifluoperazine (STELAZINE™), mesoridazine, promazine, triflupromazine (VESPRIN™), levomepromazine (NOZINAN™), promethazine (PHENERGAN™), thioxanthene (e.g., chlorprothixene, flupenthixol (DEPIXOL™, FLUANXOL™), thiothixene (NAVANE™), zuclopenthixol (CLOPIXOL™, ACUPHASE™), clozapine (CLOZARIL™), olanzapine (ZYPREXA™), risperidone (RISPERDAL™, RISPERDAL CONSTA™), quetiapine (SEROQUEL™), ziprasidone (GEODON™), amisulpride (SOLIAN™), asenapine, paliperidone, Aripiprazole (ABILIFY™), dopamine partial agonists (BIFEPRUNOX™, NORCLOZAPINE™(ACP-104)), lamotrigine (LAMICTAL™), memantine (AXURA™, AKATINOL™, NAMENDA™, EBIXA™, ABIXA™), tetrabenazine (NITOMAN™, XENAZINE™), cannabidiol, LY2140023, and the like.

Alternatively, the additional therapeutic agent can be an antidepressant and/or mood stabilizer. In certain embodiments the antidepressant comprises a monoamine oxidase inhibitor (MAOI), a tricyclic antidepressant (TCA such as IMIPRAMINE™),a tetracyclic antidepressant (TeCA), a selective serotonin reuptake inhibitor (SSRI), a noradrenergic and specific serotonergic antidepressant (NASSA), a norepinephrine (noradrenaline) reuptake inhibitor, a norepinephrine-dopamine reuptake inhibitor, and/or a serotonin-norepinephrine reuptake inhibitor (SNRI). Exemplary SSRIs include fluoxetine (PROZAC™), paroxetine (PAXIL™, SEROXAT™, escitalopram (LEXAPRO™, ESIPRAM™), citalopram (CELEXA™), sertraline (ZOLOFT™), fluvoxamine (LUVOX™)). Exemplary SNRIs include venlafaxine (EFFEXOR™), milnacipran and duloxetine (CYMBALTA™). Additional antidepressant include a noradrenergic and specific serotonergic antidepressant (NASSA) (e.g., mirtazapine (AVANZA™, ZISPIN™, REMERON™), or mianserin), a norepinephrine (noradrenaline) reuptake inhibitor (NRI) (e.g., reboxetine (EDRONAX™)), a norepinephrine-dopamine reuptake inhibitors (e.g., bupropion (WELLBUTRIN™, ZYBAN™)), Amitriptyline, Nortriptyline, Protriptyline, Desipramine, Trimipramine, Amoxapine, Bupropion, Bupropion SR, S-Citalopram, Clomipramine, Desipramine, Doxepin, Isocarboxazid, Velafaxine XR, Tranylcypromine, Trazodone, Nefazodone, Phenelzine, Lamatrogine, Lithium, Topiramate, Gabapentin, Carbamazepine, Oxcarbazepine, Valproate, Maprotiline, Mirtazapine, Brofaromine, Gepirone, Moclobemide, isoniazid, iproniazid, and the like.

In other examples, the additional therapeutic agent can be an agent for the treatment of ADD and/or ADHD. Suitable ADHD medications include, but are not limited to Statins, Amphetamine, Modafinil, Desoxyn, Methamphetamine, cocaine, arecoline, Dexmethylphenidate (Focalin, Focalin XR), dextroamphetamine (Dexedrine, Dexedrine Spansules, Dextroamphetamine ER, Dextrostat), methylphenidate (Concerta, Daytrana, Metadate CD, Metadate ER, Methylin, Methylin ER, Ritalin, Ritalin-LA, Ritalin-SR), lisdexamfetamine dimesylate (Vyvanse), mixed salts amphetamine (Adderall, Adderall XR), Atomoxetine (Strattera), clonidine hydrochloride (Catapres), guanfacine hydrochloride (Tenex), arecoline, and Pemoline.

Further, the additional therapeutic agent may be an agent for use in treating a cognitive disorder, and/or a condition characterized by neurodegeneration (e.g., Alzheimer's disease, or Parkinson's disease). Such therapeutic agents include, but are not limited to Donepezil (Aricept™), Tacrine, Rivastigmine, memantine (AXURA™, AKATINOL™, NAMENDA™, EBIXA™, ABIXA™), physostigmine, nicotine, arecoline, huperzine alpha, selegiline, Rilutek™(riluzole), vitamine C, vitamine E, carotenoids, Ginkgo biloba extract, and the like.

Exemplary additional therapeutic agent include, but are not limited to, cariprazine, brexpiprazole, butyrophenone, phenothiazine, chlorpromazine, fluphenazine, perphenazine, prochlorperazine, thioridazine, trifluoperazine, mesoridazine, promazine, triflupromazine, levomepromazine, promethazine, thioxanthene, chlorprothixene, flupenthixol, thiothixene, zuclopenthixol, clozapine, olanzapine, risperidone, quetiapine, ziprasidone, amisulpride, asenapine, paliperidone, aripiprazole, lamotrigine, tetrabenazine, cannabidiol, LY2140023, droperidol, pimozide, butaperazine, carphenazine, remoxipride, piperacetazine, sulpiride, acamprosate, vilazodone, levomilnacipran, vortioxetine, fluoxetine, paroxetine, escitalopram, citalopram, sertraline, fluvoxamine, venlafaxine, milnacipran, duloxetine, mirtazapine, mianserin, reboxetine, bupropion, amitriptyline, nortriptyline, protriptyline, desipramine, trimipramine, amoxapine, clomipramine, desipramine, doxepin, isocarboxazid, tranylcypromine, trazodone, nefazodone, phenelzine, lamatrogine, lithium, topiramate, gabapentin, carbamazepine, oxcarbazepine, valproate, maprotiline, mirtazapine, brofaromine, gepirone, moclobemide, isoniazid, iproniazid, a statin, an amphetamine, modafinil, desoxyn, methamphetamine, cocaine, arecoline, dexmethylphenidate, dextroamphetamine, methylphenidate, lisdexamfetamine dimesylate, mixed salts amphetamine, atomoxetine, clonidine hydrochloride, guanfacine hydrochloride, arecoline, pemoline, donepezil, tacrine, rivastigmine, memantine, physostigmine, lithium salts, nicotine, huperzine alpha, selegiline, riluzole, vitamin C, vitamin E, carotenoids, and Ginkgo biloba extract.

In aspect of toxicology, glycine is used as food additive for seasoning. It is reported that NOAEL (no-observed-adverse-effect level) of glycine is at least 2000 mg/kg/day in rat, and LD50 (lethal dose, 50%) of glycine is 7930 mg/kg in rat. The acute oral LD50 of phosphatidylserine is greater than 5 g/kg bw and NOAEL of phosphatidylserine is 1,000 mg/kg bw/day in rats and dogs. In addition, it is reported that LD50 of coumarin is 290 mg/kg in rat, LD50 of cinnamic acid is>5000 mg/kg in rat, mouse, and guinea pig, LD50 of cinnamaldehyde is 3400 mg/kg in rat, mouse, and guinea pig, and LD50 of cinnamyl alcohol is 2675 mg/kg in rat, mouse, and guinea pig. Accordingly, the dosage of glycine or phosphatidylserine combined with cinnamon extract in the composition, the method, and the combination according to the present disclosure is safe for animals, and the composition according to the present disclosure has great potential to be health food, health supplement and food additive for improving sense, mood, sleeping behavior, anxiety, depression, concentration, attention, cognition, longevity, thought, or memory.

### EXPERIMENTAL EXAMPLES

### Example 1. Determination of Constitutions in Cinnamon Extracts

### Instruments and Chemicals

The HPLC analysis was performed by using a SHIMADZU system equipped with a LC-6 AD binary HPLC pump and a SPD-20 A UV/Vis detector, and SIL-20 A liquid handler autosampler injector. Column temperature was set at room temperature. HPLC-grade reagents were water containing 0.1% formic acid, and acetonitrile. Three constituents, including coumarin (CM), cinnamic acid (CA), and trans-cinnamaldehyde (TCAL), were selected as standard compounds.

The standard compounds of CM, CA, and TCAL were weighed and dissolved in 50% EtOH to obtain a stock standard (1.00 mg/mL). The aqueous solutions were prepared in concentrations of 0.24, 0.49, 0.98, 1.95, 3.91, 7.81, 15.63, 31.25, 62.50, 125.00, and 250.00 μg/mL for the calibration curve. The cinnamon bark extracts were purchased from Supplier A (Cinnamomum cassia from China), Supplier B (Cinnamomum cassia from China), Supplier C (Cinnamomum cassia from India), and Supplier D (Cinnamomum Burmannii from China). The cinnamon leaf powder was purchased from Supplier E (Cinnamomum osmophloeum from Taiwan). 20.00 mg of each cinnamon bark extract or cinnamon leaf powder was dissolved in 1 mL of 50% EtOH and extracted at 50° C. for 2 hours and was filtered with a 0.45 μm syringe polyvinylidene difluoride (PVDF) filter. The dissolved solutions were used for HPLC analysis.

### HPLC Conditions and Calibration Curves

HPLC analysis was conducted using a Phenomenex Luna Omega C₁₈ (4.6×150 mm, 5 μm) column. Gradient flows for the two solvent system (solvent A, 0.1% formic acid in water; solvent B, acetonitrile) were as follows: 0 min, 20% B; 35 min, 50% B; 40 min, 100% B; and hold at 100% B for 5 min; 46 min, 20% B; and hold at 20% B for 9 min. The flow rate of the mobile phase was 1.0 mL/min. UV detection was conducted at an absorbance of 280 nm. The injection volume was 30 μL.

The standard solution of CM, CA, and TCAL (0.24-250.00 μg/mL) were prepared in 50% EtOH. The CM, CA, and TCAL contents of the cinnamon samples were determined from the corresponding calibration curves. The calibration functions of CM, CA, and TCAL were calculated by using the peak area (Y) and concentration (X, μg/mL). The range of the calibration curves of coumarin (CM), cinnamic acid (CA) and trans-cinnamaldehyde (TCAL) are 0.24-250.00 μg/mL, 0.24-125.00 μg/mL and 0.24-125.00 μg/mL, separately.

### Results

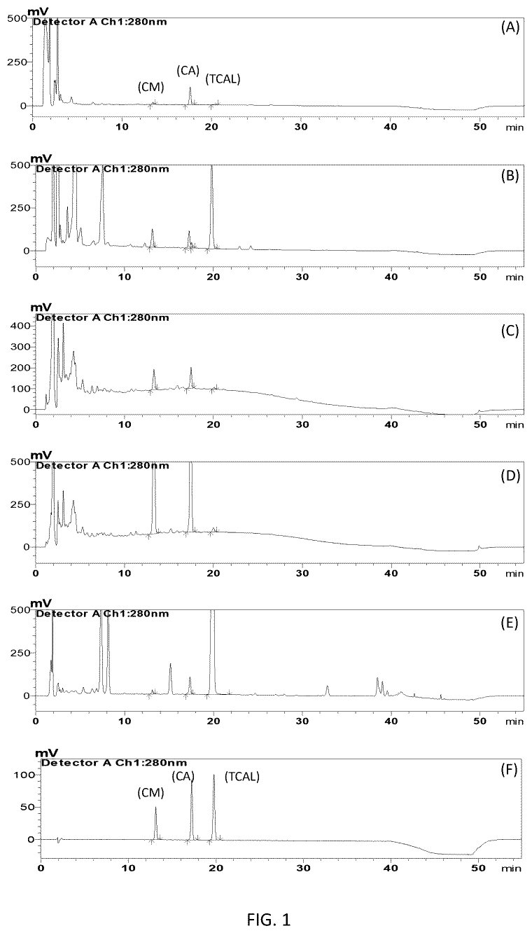

The CM, CA, and TCAL contents of cinnamon bark extracts and cinnamon leaf powder from different suppliers were shown in Table 1 and FIG. 1. FIG. 1 shows HPLC chromatograms of cinnamon bark extracts purchased from (A) Supplier A (Cinnamomum cassia from China), (B) Supplier B (Cinnamomum cassia from China), (C) Supplier C (Cinnamomum cassia from India), and (D) Supplier D (Cinnamomum Burmannii from China), and cinnamon leaf powder purchased from (E) Supplier E (Cinnamomum osmophloeum from Taiwan), and HPLC chromatogram of (F) mixed standard solution of CM, CA and TCAL (15.63 μg/mL). Peaks: (CM) coumarin, (CA) cinnamic acid, and (TCAL) trans-cinnamaldehyde.

TABLE 1
The CM, CA, and TCAL contents of cinnamon bark extracts
and cinnamon leaf powder from different suppliers.

| | Extract or | | County of | | Concentration |
|---|---|---|---|---|---|
| Supplier | powder | Source plant | origin | Compound* | (mg/g) |
| A | bark extract | Cinnamomum | China | CM | 0.05 |
| | | cassia | | CA | 0.31 |
| | | | | TCAL | 0.01 |
| B | bark extract | Cinnamomum | China | CM | 1.39 |
| | | cassia | | CA | 0.65 |
| | | | | TCAL | 3.47 |
| C | bark extract | Cinnamomum | India | CM | 0.62 |
| | | cassia | | CA | 0.32 |
| | | | | TCAL | 0.02 |
| D | bark extract | Cinnamomum | China | CM | 7.56 |
| | | Burmannii | | CA | 4.14 |
| | | | | TCAL | 0.08 |
| E | leaf powder | Cinnamomum | Taiwan | CM | 0.07 |
| | | osmophloeum | | CA | 0.31 |
| | | | | TCAL | 13.94 |

### Example 2. Therapeutic Effectiveness of NMDA Modulator Combined With DAAO Inhibitor and NMDA Modulator Combined With Cinnamic Extract in the Cognitive Deficit Analyzed by the Novel Object Recognition Test

The objective of the Novel Object Recognition (NOR) Test was to investigate the therapeutic effectiveness of the combination of NMDA modulator with DAAO inhibitor and the combination of NMDA modulator with cinnamic extract in alleviating the cognitive deficit, a common symptom of most neurological disorders. MK-801, a NMDA antagonist, was used to generated cognitive impaired model for comparing the therapeutic effectiveness of different formulations, NMDA modulator alone, DAAO inhibitor alone, and NMDA modulator combined with DAAO inhibitor for the first part; and NMDA modulator, cinnamic extract alone, and NMDA modulator combined with cinnamic extract for the second part. The testing compounds and MK-801 were administrated in mice, respectively, by oral gavage and intraperitoneal (i.p.) injections before the novel object recognition test.

### Materials and Methods

C57BL/6J male mice were group housed (3-5 mice per cage) with food and water available ad libitum in polysulfone ventilated cages (Alternative Design, AR, USA) in the animal rooms. The colony was maintained on a 12/12-h light/dark cycle at the temperature of 22±2° C. and all behavioral studies will be performed during the dark cycle. All animals used in this experiment were adult mice (at least 8 weeks of age). All animal procedures were performed according to the protocols approved by Institutional Animal Care and Use Committee (IACUC).

For the first part, the mice were randomly assigned into eight groups listed below:
Group 1: Vehicle control, n=10
Group 2: MK-801 alone, n=19
Group 3: NMDA modulator (50 mg/kg), n=3
Group 4: DAAO inhibitor (50 mg/kg), n=3
Group 5: NMDA modulator (50 mg/kg)+DAAO inhibitor (50 mg/kg), n=9
Group 6: NMDA modulator (100 mg/kg), n=21
Group 7: DAAO inhibitor (100 mg/kg), n=8
Group 8: NMDA modulator (100 mg/kg)+DAAO inhibitor (100 mg/kg), n=9

In the first part, mice in Groups 2-8 received an acute dose of MK-801 (Sigma-Aldrich USA), a NMDA receptor antagonist, dissolved in normal saline, at 0.2 mg/kg by i.p. injection 30 minutes prior to novel object recognition test. Each mouse in Groups 3-8 orally received an acute dose of NMDA modulator, DAAO inhibitor, or NMDA modulator combined with DAAO inhibitor (dissolved in 50% PEG) 30 minutes prior to the MK-801 administration. The cognitive performance of each group was tested by novel object recognition test.

For the second part, the mice were randomly assigned into five groups listed below:
Group 1: Vehicle control, n=10
Group 2: MK-801 alone, n=19
Group 3: NMDA modulator (100 mg/kg), n=21
Group 4: Cinnamon extract (100 mg/kg), n=9
Group 5: NMDA modulator (100 mg/kg)+Cinnamon extract (100 mg/kg), n=11

In the second part, mice in Groups 2-5 received an acute dose of MK-801 (Sigma-Aldrich USA), a NMDA receptor antagonist, dissolved in normal saline, at 0.2 mg/kg by i.p.

injection 30 minutes prior to novel object recognition test. Each mouse in Groups 3-5 orally received an acute dose of NMDA modulator, cinnamon extract, or NMDA modulator combined with cinnamon extract (dissolved in 50% PEG) 30 minutes prior to the MK-801 administration. The cognitive performance of each group was tested by novel object recognition test.

The novel object recognition test is a widely used task for assessing the cognitive performance in rodent models. The task was based on the spontaneous alternation, a natural tendency of rodents to seek different stimuli in consecutive trails. In the first trail, mice were allowed to explore a square arena (40×40×40; Unit: cm) under 15-20 lux light intensity with two identical objects (Yellow square Lego bricks) for 5 min and were then moved to the home cage for 5 min. In the second trial, one of two previously explored object was change to a novel object (Blue circular bottle cap). Mice was placed to the arena and allowed to explore for 10 min. Normal animals show high tendency to explore the novel object with higher *discrimination index, compared with cognition-impaired animals.

$$Discrimination index = {Time}_{( novel object )} - {Time}_{( explored object )} / Total exploring time$$

### Results

The first part (NMDA modulator combined with DAAO inhibitor):

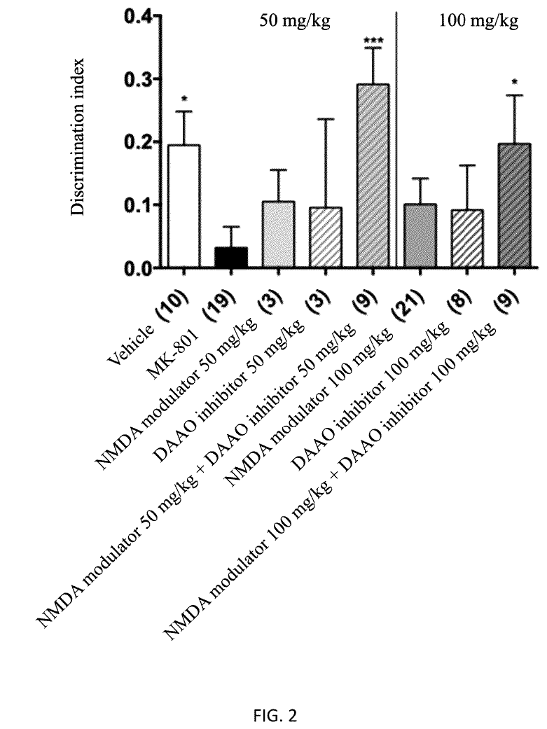

FIG. 2 shows the discrimination index of novel object recognition test in MK801-treated mice received acute administration (50 or 100 mg/kg) of NMDA modulator alone, DAAO inhibitor alone, or NMDA modulator combined with DAAO inhibitor. Data were presented as mean±SEM and analyzed with Student's T-test. * P<0.05, ***P<0.001, compared with MK-801 alone group. FIG. 2 displays the superior therapeutic effect of NMDA modulator+DAAO inhibitor combination in alleviating MK-801-induced cognitive impairment. All tested formulations could increase discrimination index of MK-801-treated mice in the novel object recognition test. The combination of NMDA modulator and DAAO inhibitor consistently exhibited enhanced therapeutic effectiveness in alleviating cognitive impairment, compared with the equivalent doses of NMDA modulator and DAAO inhibitor alone (50 or 100 mg/kg). The NMDA modulator+DAAO inhibitor-treated mice exhibited significantly higher discrimination index (Group 5: P<0.001; Group 8: P<0.05, respectively; Student's t-test), compared with MK-801 alone group. This result indicates that the combination of NMDA modulator and DAAO inhibitor is more potent than NMDA modulator or DAAO inhibitor alone and has great potential as a novel medication, health supplement, or bioactive substance for cognitive impairment.

The second part (NMDA modulator combined with cinnamic extract):

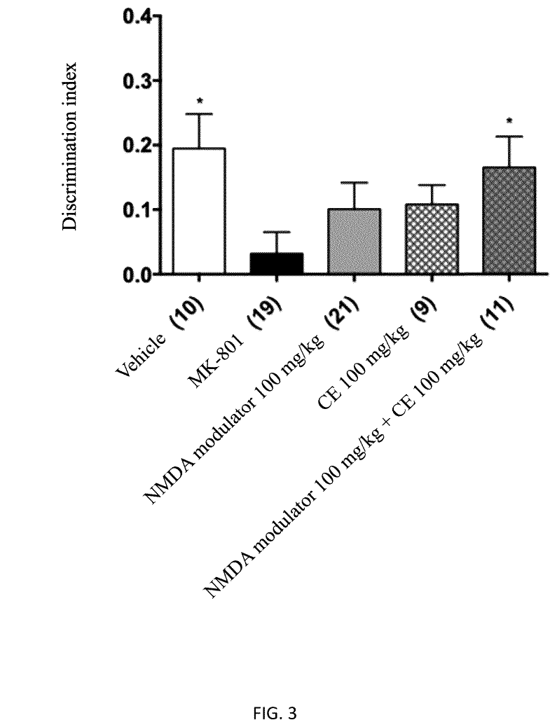

FIG. 3 shows the discrimination index of novel object recognition test in MK801-treated mice received acute administration of NMDA modulator, cinnamon extract (CE) or NMDA modulator combined with cinnamon extract. Data were presented as mean±SEM and analyzed with Student's T-test. * P<0.05, compared with MK-801 alone group. FIG. 3 displays the superior therapeutic effect of NMDA modulator+cinnamon extract combination in alleviating MK-801-induced cognitive impairment. All three formulations (Groups 3, 4, and 5) could alleviate MK-801-induced cognitive impairment. The combination of NMDA modulator with cinnamon extract showed higher potency in alleviating MK-801-induced cognitive impairment compared with NMDA modulator or cinnamon extract alone formulation. The NMDA modulator+cinnamon extract-treated mice (Group 5) exhibited significantly higher discrimination index (P<0.05; Student's t-test), compared with MK-801 group. This result indicates that the combination of NMDA modulator and cinnamon extract is more potent than NMDA modulator or cinnamon extract alone and has great potential as a novel medication, health supplement, or bioactive substance for cognitive impairment.

### Example 3. Therapeutic Effectiveness of NMDA Modulator Combined With DAAO Inhibitor Analyzed by Forced Swimming Test

The objective of Forced Swimming Test (FST) is to investigate the anti-depressive effectiveness of the formulation that combines NMDA modulator and DAAO inhibitor. Forced swimming test (FST), a rodent behavioral study, was used in this experiment for assessing the anti-depressive efficacy of different formulations, NMDA modulator alone, DAAO inhibitor alone, and NMDA modulator+DAAO inhibitor. In FST test, mice were placed in inescapable circular tank filled with water and their escaping latencies were measured as an indicator of anti-depressive effectiveness.

### Materials and Methods

This part is the same as example 2.

The mice were randomly assigned into four groups listed below:
Group 1: Vehicle Control, n=8
Group 2: NMDA modulator (100 mg/kg), n=5
Group 3: DAAO inhibitor (100 mg/kg), n=7
Group 4: NMDA modulator (100 mg/kg)+DAAO inhibitor (100 mg/kg), n=8

All tested mice were orally received an acute dose of control vehicle (Group 1, saline) or test groups (Group 2-4, dissolved in ddH2O) 30 min prior to the FST.

### Procedures of FST:

FST is a learned helplessness model, which is widely used in evaluating the effectiveness of anti-depressants in rodent models. The apparatus of FST is a glass cylinder tank (Diameter: 12 cm×Height: 40 cm) filled with water level 25 cm from the bottom. This tank creates an inescapable environment and results in a stress of drowning. The response, particular immobility or unwilling to floating, of tested animals could be interpreted as the susceptibility of negative mood or stress. In this experiment, one-trail protocol was used for assessing the effectiveness of anti-depressants, which mice were placed on the cylinder tank for swimming or floating for 6 min trial. Inactivity or the movement for balancing the body or maintain the head above the water was considered as immobility of FST test. Only the movement of the last 4 min was evaluated due to the hyperactivity of rodents in the first 2 min that might underestimate the immobility of FST.

### Results

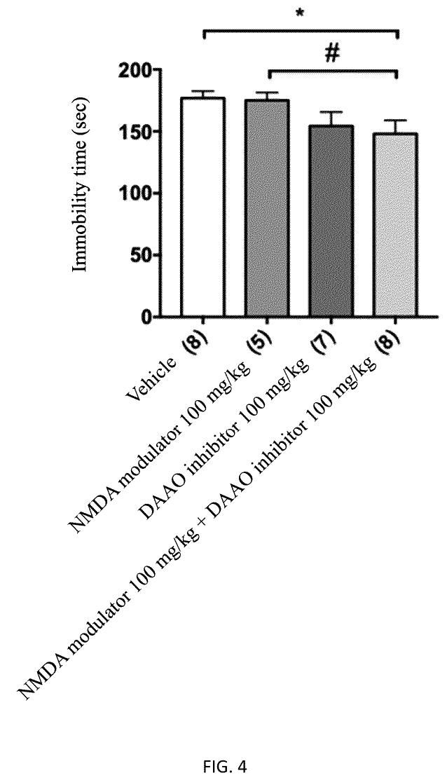

FIG. 4 shows the immobility of forced-swimming test in control and test-groups, including NMDA modulator, DAAO inhibitor, and NMDA modulator+DAAO inhibitor-treated mice. Combination treatment significantly reduces the immobility time in FST, an indicator of depressed moods, compared with the vehicle group (*P<0.05) and NMDA modulator-treated (#P<0.05) group in this experiment. FIG. 4 displays the superior anti-depressive effect of NMDA modulator combined with DAAO inhibitor in the FST test. In this experiment, two tested formations, DAAO inhibitor alone and NMDA modulator+DAAO inhibitor, show anti-depressive effect which reduces the immobility of escaping latency in FST test. NMDA modulator+DAAO inhibitor-treated mice exhibit the lowest immobile time in all tested compounds of the FST test and this result indicates the enhanced anti-depressive potency of the formulation, NMDA modulator+DAAO inhibitor, compared with the equivalent doses of NMDA modulator or DAAO inhibitor alone. Overall, NMDA modulator+DAAO inhibitor is a potent anti-depressant and has a great potential as a novel medication for depression due to major depression or other psychiatric diseases.

### Example 4. Therapeutic Effectiveness of NMDA Modulator Combined With DAAO Inhibitor on Prepulse Inhibition Test

Therapeutic effectiveness of NMDA modulator combined with DAAO inhibitor in alleviating impaired prepulse inhibition (PPI) was demonstrated in this experiment, with a mouse model having such neuropsychiatric symptom induced by MK-801. The test compounds and MK-801 were administrated to mice, respectively, by oral gavage and intraperitoneal (i.p.) injections. The startle response measured in PPI test was used as an indicator of therapeutic effect of test compounds in this experiment.

### Materials and Methods

This part is the same as example 2.

The mice were randomly assigned into four groups listed below:
Group 1: Vehicle control, n=9
Group 2: MK-801 alone, n=11
Group 3: NMDA modulator (100 mg/kg)+DAAO inhibitor (100 mg/kg), n=6

Prepulse inhibition, using SR-LAB startle apparatus (San Diego Instruments, San Diego, CA, USA), was used to determine the efficacy of test compounds (Groups 3 and 4) on attenuating the MK-801 (0.3 mg/kg)-induced deficit of sensorimotor gating in mice. Under 65 dB background noise, each session was composed of a 5-minute accumulation period followed by 64 trials in four blocks. The initial and the last blocks were composed of six pulse alone (PA) trials. Two middle blocks consisted of PA, prepulse (pp)+pulse, and non-stimulus (NS) trials. The pulse alone (PA) trial was a 40 ms, 120 dB white noise burst. In the prepulse (pp)+pulse trials, a 20 ms prepulse stimuli of 71 dB (pp6), 75 dB (pp10) or 83 dB (pp18) was presented 100 ms before a 40 ms 120 dB pulse. In non-stimulus (NS) trials, only background noise was presented. These trials were presented pseudo-randomly and separated by intertribal intervals of 15 seconds on average (varying between 10 to 20 s). The percentage of prepulse inhibition was evaluated by the following formula: % PPI=100×[(PA score)−(pp-P score)]/(PA score), while the PA score was the average of the PA value in the middle blocks.

### Results

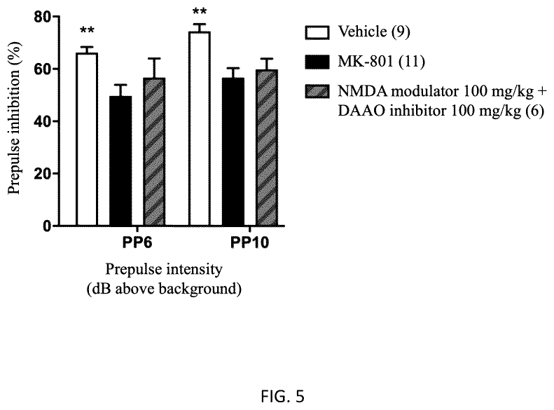

FIG. 5 shows the effects of NMDA modulator combined with DAAO inhibitor on prepulse inhibition test in MK-801 (0.3 mg/kg; intraperitoneal injection)-treated mice. Data were presented as mean±SEM and analyzed with Student's T-test. **P<0.001, compared with MK-801 alone group. FIG. 5 displays the therapeutic effect of NMDA modulator combined with DAAO inhibitor on PPI impairment of MK-801-treated mice. NMDA modulator combined with DAAO inhibitor could alleviate MK-801 induced PPI deficit at PP6 and PP10. This result indicates that NMDA modulator in combination with DAAO inhibitor has a great potential as novel medications for neuropsychiatric diseases.

In view of the above description and experimental examples, the composition according to the present disclosure can improve cognitive performance and have anti-depression effectiveness, which are demonstrated in the novel object recognition test, the forced swimming test, and the prepulse inhibition test. From these experimental results, the composition according to the present disclosure has great potential to be health food, health supplement and food additive for improving sense, mood, sleeping behavior, anxiety, depression, concentration, attention, cognition, longevity, thought or memory, and is expected to be a medication for treating, preventing, or delaying psychiatric disorders and CNS disorders.

### Example 5: The Contents of N-methyl-D-aspartate Receptor Modulators in Probiotics

The purpose of this study is to determine the contents of N-methyl-D-aspartate receptor (NMDAR) modulators, selecting from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, DL-cycloserine, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and tumeric extract in probiotics.

### Materials and Methods

The powder of probiotics was dissolved in the solution (50% methanol/water) and sonicated for 15 min for extraction. Then, the solution was centrifuged at 12,000 rpm for 10 min. The supernatant was filtered by 0.22 μm filter and analyzed by LC-MS/MS with the following method. An Agilent 1260 LC quaternary pump with Agilent 1260 LC infinity autosampler and Agilent 1100 column oven (Marshall Scientific, Hampton, NH, USA) were used to inject the samples onto an Astec Chirobiotic column (5 μm, 250×4.6 mm I.D., Sigma). The mobile phase was a 20/80 mixture of mobile phase A (0.1% formic acid in ddH₂O) and mobile phase B (0.1% formic acid in acetonitrile) and the run was isocratic at a flow rate of 0.3 ml/min. Quantitation was achieved by MS/MS detection in positive ion mode for analyte and internal standard using a AB SCIEX 3200 triple quadrupole ion trap mass spectrometer.

### Results

Our results show that NMDA modulators are enriched in different stains of probiotics. Two strains, Lactobacillus plantarum and Lactobacillus acidophilus, has high contents of NMDAR modulators in this study. The findings of our study revealed that probiotics can served as source of NMDA modulators for improving the cognitive function of patients with psychiatric disorders.

TABLE 2
presents the different contents of N-methyl-D-aspartate
receptor modulators* in different species of probiotics.

| | | The concentration of NMDA |
|---|---|---|
| Strain | CFU/g | modulators (μg/10¹¹ CFU) |
| Lactobacillus plantarum | 4 × 10¹¹ | 547.64 ± 21.67 |
| Lactobacillus rhamnosus | 2 × 10¹¹ | 187.21 ± 0.17 |
| Lactobacillus acidophilus | 1 × 10¹¹ | 455.17 ± 2.09 |
| Bifidobacterium longum | 2 × 10¹¹ | 63.45 ± 18.66 |

*NMDA modulators select from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, DL-cycloserine, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and tumeric extract.

### Example 6: Enhanced Sedative Efficacy of NMDA Modulators in Combination With D-Amino Acid Oxidase Inhibitors

The objective of this experiment was to investigate the sedative effects of NMDA modulators (NMDAM) in combination with D-amino acid oxidase inhibitors (DAAOI). The test compounds were administered to mice via oral gavage prior to the open field test. Locomotor activity was assessed by measuring the total number of beam breaks during the open field test, which served as an indicator of both locomotor function and the sedative effects of the test compounds.

### Materials and Methods

The mice were randomly assigned into two groups listed below:
Group 1: Vehicle control n=7
Group 2: NMDAM+DAAOI n=8

The open field task is a common measurement of novelty induced exploratory behavior and general activity in both mice and rats. In this study, the mice were placed in a Plexiglas cage (37.5 cm×21.5 cm×18 cm) under 50-65 lux light intensity. Their spontaneous locomotor activities were measured for 60 minutes using the Photobeam Activity System (PAS)-open field (San Diego Instruments, San Diego, CA, USA). The total number of photo beam breaks of each mouse was measured as an index of locomotor activity.

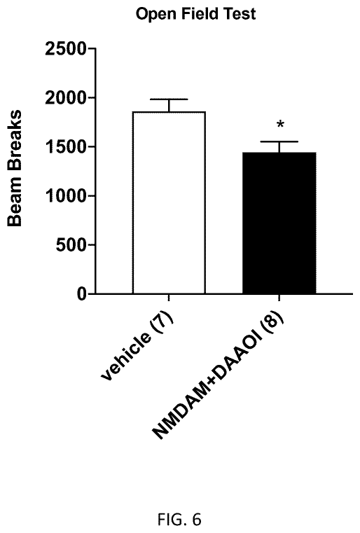

FIG. 6 displays the superior sedative effect of NMDAM combined with DAAOI. Co-administration of NMDAM and DAAOI resulted in a significant reduction (P<0.05, student t-test) in locomotor activity, with treated mice showing up to a 22% decrease in beam breaks in the open field test compared to controls.

### Example 7: Therapeutic Efficacy of NMDA Modulators Combined With D-Amino Acid Oxidase Inhibitors in Cognitive Impairment

The objective of this experiment was to investigate the therapeutic effectiveness NMDA modulators (NMDAM) combined with D-amino acid oxidase inhibitors (DAAOI) in alleviating the cognitive deficit, a common symptom of most neurological disorders and compared with NMDAM or DAAOI alone. MK801, a NMDA antagonist, was used to generated cognitive impaired model for potency comparing. The testing compounds and MK-801 were administrated in mice, respectively, by oral gavage and intraperitoneal (i.p.) injections before the novel object recognition tests.

### Materials and Methods

The mice were randomly assigned into 18 groups listed below:
Group 1: vehicle control; Group 2: MK-801; Group 3: Sodium Benzoate (NaBen; 12mg/kg)+MK-801; Group 4: NaBen (12 mg/kg)+Sarconsine (SAR; 600 mg/kg)+MK-801;
Group 5: D-cycloserine (DCS; 12 mg/kg)+MK-801; Group 6: DCS (12 mg/kg)+Lithium Benzoate(LiBen 550 mg/kg)+MK-801; Group 7: D-alanine (D-Ala; 100 mg/kg)+MK-801;
Group 8: Cinnamic acid (CA; 100 mg/kg)+MK-801; Group 9: D-Ala (100 mg/kg)+CA (100 mg/kg) MK-801; Group 10: Cinnamon extract (CE; 100 mg/kg) MK-801; Group 11: D-Ala (100 mg/kg)+CE(100 mg/kg) MK-801; Group 12: Phenylalanine (Phe, 12 mg/kg)+MK-801 Group 13: Phe (12 mg/kg)+NaBen (600 mg/kg) +MK-801 Group 14: Glycine (Gly, 12 mg/kg)+MK-801 Group 15: Glycine (Gly, 12 mg/kg)+NaBen (600 mg/kg)+MK-801 Group 16: Taurine (Tau, 8 mg/kg)+Tannic acid (TA, 400 mg/kg)+MK-801 Group 17: Nicotinic acid (Nic, 8 mg/kg) +MK-801 Group 18: Nic(20 mg/kg) +DL-serine (Ser, 500 mg/kg)+MK-801

The novel object recognition test is a widely used task for assessing the cognitive performance in rodent models. The task was based on the spontaneous alternation, a natural tendency of rodents to seek different stimuli in consecutive trails. In the first trail, mice were allowed to explore a square arena (40×40×40; Unit: cm) under 15-20 lux light intensity with two identical objects (Yellow square Lego bricks) for 5 min and were then moved to the home cage for 5 min. In the second trial, one of two previously explored object was change to a novel object (Blue circular bottle cap). Mice was placed to the arena and allowed to explore for 10 min. Normal animals show high tendency to explore the novel object with higher *discrimination index, compared with cognition-impaired animals.

$$Discrimination index = {Time}_{( novel object )} - {Time}_{( explored object )} / Total exploring time$$

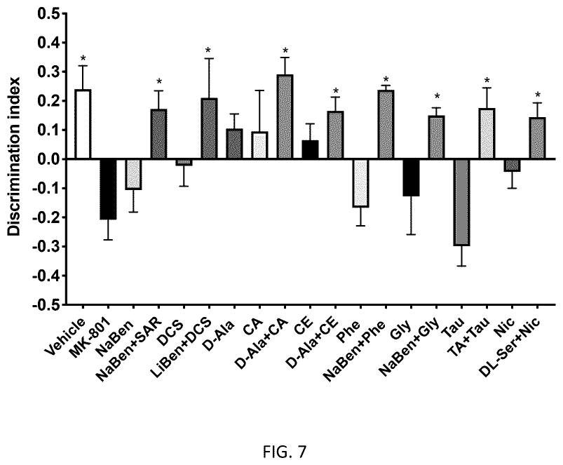

FIG. 7 displays superior therapeutic effects of combined NMDAM and DAAOI treatment on cognitive impairment. The combined treatment groups demonstrated significantly greater and unexpected improvements in cognitive function compared to MK-801 and monotherapy groups (P<0.05; one-way ANOVA followed by post hoc Dunnett's test).

Example 8: Enhanced Therapeutic Effectiveness of NMDA Modulator in Combination with D-Amino Acid Oxidase Inhibitors on Sleeping Disturbances

The objective of this experiment was to investigate the therapeutic effects of an NMDA modulator (NMDAM) in combination with D-amino acid oxidase inhibitors (DAAOI). Participants were assessed using the Pittsburgh Sleep Quality Index (PSQI) to screen for individuals with mild to moderate sleep disturbances, defined as a PSQI score between 6 and 9.

Eligible participants received oral administration of NMDAM combined with DAAOI one hour before bedtime for four weeks. Following the treatment period, the PSQI was re-administered to evaluate changes in sleep quality.

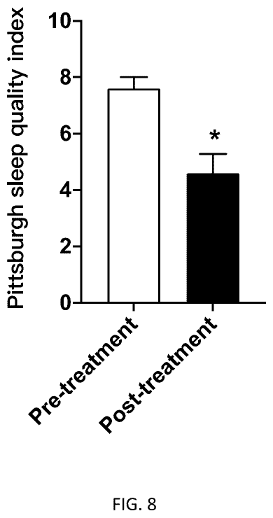

FIG. 8 displays superior therapeutic effects of the NMDA modulator (NMDAM) combined with a D-amino acid oxidase inhibitor (DAAOI) on sleep disturbances. Treatment with NMDAM and DAAOI over four weeks led to a significant reduction in PSQI scores (P <0.05, Student's t-test), indicating an improvement in sleep quality.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above

Description, but rather is as set forth in the appended claims. Those of ordinary skill in the art will appreciate that various changes and modifications to this description may be made without departing from the spirit or scope of the present invention, as defined in the following claims.

What is claimed is:

1. A composition, comprising:
a D-amino acid oxidase (DAAO) inhibitor; and
a N-methyl-D-aspartate (NMDA) modulator.

2. The composition of claim 1, wherein the DAAO inhibitor is selected from a group consisting of cinnamon extract, sodium benzoate, lithium benzoate, benzoic acid, sorbic acid, cinnamic acid, and tannic acid.

3. The composition of claim 1, wherein the NMDA modulator is selected from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, DL-cycloserine, a probiotic composition, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and tumeric extract.

4. The composition of claim 2, wherein the cinnamon extract is extracted from bark or leaf of Cinnamomum cassia, Cinnamomum austrosinense, Cinnamomum brevipedunculatum, Cinnamomum burmannii, Cinnamomum insularimontanum, Cinnamomum kotoense, Cinnamomum macrostemon, Cinnamomum osmophloeum, Cinnamomum reticulatum, Cinnamomum sericeum, Cinnamomum subavenium, Cinnamomum temufolium nervosum, Cinnamomum verum, or Cinnamomum loureiroi.

5. The composition of claim 3, a probiotic composition comprising a plurality of probiotic strains selected from the group consisting of Lactobacillus acidophilus, Lactobacillus casei, Lactobacillus rhamnosus, Lactobacillus plantarum, Lactobacillus reuteri, Lactobacillus fermentum, Lactobacillus salivarius, Lactobacillus bulgaricus, Lactobacillus brevis, Lactobacillus paracasei, Lactobacillus gasseri, Lactobacillus johnsonii, Bacillus coagulans, Bifidobacterium adolescentis, Bifidobacterium animalis subsp. lactis, Bifidobacterium bifidum, Bifidobacterium breve, Bifidobacterium longum, Bifidobacterium infantis, Bifidobacterium lactis, Bifidobacterium pseudocatenulatum, Bifidobacterium thermophilum, Bifidobacterium pseudolongum or Clostridium buyricum.

6. The composition of claim 3, wherein the probiotic count is 1×10⁶ to 1×10¹² CFU.

7. The composition of claim 3, wherein the probiotic count is 1×10⁹ to 1×10¹¹ CFU.

8. The composition of claim 1, further comprising a pharmaceutically acceptable excipient, carrier, diluent, binder, additive, filler, lubricant, nutrient, or a mixture thereof.

9. The composition of claim 1, wherein the weight ratio of the DAAO inhibitor to the NMDA modulator is 1:100 to 100:1.

10. The composition of claim 1, wherein the weight ratio of the DAAO inhibitor to the NMDA modulator is 1:50 to 50:1.

11. The composition of claim 10, wherein the DAAO inhibitor is selected from the group consisting of cinnamon extract, sodium benzoate, cinnamic acid, and tannic acid, the NMDA modulator is selected from a group consisting of glycine, sarcosine, dimethylglycine (DMG), DL-serine, DL-alanine, and DL-cycloserine, and the weight ratio of the DAAO inhibitor to the NMDA modulator is 1:50 to 50:1.

12. The composition of claim 10, wherein the DAAO inhibitor is selected from the group consisting of cinnamon extract, sodium benzoate, cinnamic acid, and tannic acid, the NMDA modulator is selected from a group consisting of glycine, sarcosine, dimethylglycine (DMG), D-serine, D-alanine, and D-cycloserine, and the weight ratio of the DAAO inhibitor to the NMDA modulator is 1:50 to 50:1.

13. The composition of claim 12, wherein the DAAO inhibitor is selected from the group consisting of cinnamon extract, sodium benzoate, cinnamic acid, and tannic acid, the NMDA modulator is selected from a group consisting of glycine, sarcosine, dimethylglycine (DMG), D-serine, D-alanine, and D-cycloserine, and the weight ratio of the DAAO inhibitor to the NMDA modulator is 1:5 to 5:1.

14. The composition of claim 1, wherein the composition is a pharmaceutical composition, a nutraceutical composition, a medical food, a health food, a health supplement, or a food additive.

15. The composition of claim 1, wherein the composition is formulated in a form of tablet, pill, capsule, powder, granule, solution, suspension, soft chew, or gel.

16. A method for treating, preventing, or delaying the onset of the psychiatric disorder or the central nervous system (CNS) disorder, comprising administering an effective amount of the composition of claim 1 to a subject in need thereof.

17. The method of claim 16, wherein the psychiatric disorder is selected from a group consisting of depressive disorder, major depression, anhedonia, eating disorder, elimination disorder, feeding disorder, depression, dysthymia, mood disorder, anxiety disorder, personality disorder, addictive behavior, obsessive-compulsive and related disorder, trauma- and stressor-related disorders, dissociative disorder, sleep-wake disorder, sleep disorder, insomnia, somatic symptom and related disorder, social anxiety disorder, depression-related syndrome, symptom of depression due to a physical disorder, and drug-induced symptom of depression.

18. The method of claim 16, wherein the CNS disorder is selected from a group consisting of schizophrenia, bipolar disorder (BD), attention-deficit disorder, attention-deficit hyperactivity disorder (ADHD), obsessive compulsive disorder (OCD), Tourette's syndrome, blepharospasm, post-traumatic stress disorder (PTSD), addiction disorder, psychotic disorder, panic disorder, autism spectrum disorder (ASD), Asperger's disorder, Alzheimer's disease, mild cognitive disorder (MCI), benign forgetfulness, vascular dementia, dementia with Lewy bodies (DLB), Huntington's disease (HD), premenstrual syndrome, nocturnal enuresis, non-epileptic seizures, mild cognitive impairment, mania, learning disorder, pain, Parkinson's disease, Duchenne muscular dystrophy, stoke, closed head injury, memory deficits, frontotemporal dementia (FTD), multiple sclerosis, amyotrophic lateral sclerosis (ALS), Lyme borreliosis, tick-borne diseases (TBD), fragile X syndrome (FXS), and traumatic brain injury.

19. The method of claim 16, wherein the DAAO inhibitor of the composition of claim 1 is selected from a group consisting of cinnamon extract, sodium benzoate, lithium benzoate, benzoic acid, sorbic acid, cinnamic acid, and tannic acid.

20. The method of claim 16, wherein the NMDA modulator is selected from a group consisting of glycine, sarcosine, dimethylglycine (DMG), trimethylglycine (TMG), D-phenylalanine, L-phenylalanine, DL-phenylalanine, phosphatidylserine, D-serine, L-serine, DL-serine, D-alanine, L-alanine, DL-alanine, D-cycloserine, L-cycloserine, DL-cycloserine, a probiotic composition, taurine, nicotinamide, tryptophan, threonine, γ-aminobutyric acid (GABA), tyrosine, 5-hydroxytryptophan (5HTP), docosahexaenoic acid (DHA), eicosapentaenoic acid (EPA), lutein, lemon balm extract, and tumeric extract.
